(19) United States
(12) Patent Application Publication
(10) Pub. No.: US 2023/0398080 A1
(43) Pub. Date: Dec. 14, 2023

(54) FLOATING DRUG DELIVERY SYSTEMS COMPRISING CANNABINOIDS

(71) Applicant: YISSUM RESEARCH DEVELOPMENT COMP ANY OF THE HEBREW UNIVERSITY OF JERUSALEM LTD., Jerusalem (IL)

(72) Inventors: Michael FRIEDMAN, Jerusalem (IL); Amnon HOFFMAN, Jerusalem (IL); Dvora IZGELOV, Holon (IL)

(21) Appl. No.: 17/758,163

(22) PCT Filed: Dec. 30, 2020
(86) PCT No.: PCT/IL2020/051351

§ 371 Date: Jun. 29, 2022

Related U.S. Application Data
(60) Provisional application No. 62/956,442, filed on Jan. 2, 2020.

(51) Int. Cl. A61K 9/20; A61K 31/00; A61K 47/42

(52) U.S. Cl. CPC A61K 9/2095; A61K 31/658; A61K 47/42

(57) ABSTRACT

The present invention concerns a solid oral dosage form comprising albumin and at least one cannabinoid, wherein the oral dosage form is free of a gas generating agent (GGA) and wherein the oral dosage form is prepared using a compression force Q of below 1 ton.

### TECHNOLOGICAL FIELD

The invention disclosed herein generally concerns floating sustained-release drug delivery systems containing cannabinoids.

### BACKGROUND

It is well recognized that the stomach may be used as a ‘depot’ for sustained-release dosage forms [1]. Gastro-retentive dosage forms (GRDF) have been the topic of interest in recent years as a practical approach in drug deliveries to the upper GI tract [2]. GRDF may be highly useful for the delivery of many drugs. Some examples of drugs that can be best delivered using such dosage forms are drugs that have a narrow window of absorption, drugs that are poorly soluble in an alkaline pH, drugs that are degraded in the colon and drugs that act locally in the stomach [3].

Over the last decades, various approaches have been pursued to increase the retention of an oral dosage form in the stomach; one of these approaches is the floating drug delivery system [1]. Oral floating devices are made to be retained in the stomach for a long time assuring a slow delivery of the drug above its absorption site, thus providing increased and more reproducible bioavailability [4]. These floating devices can also be used in a local treatment of gastric pathologies.

Generally, floating dosage forms can be divided into two broad types: gas-releasing and non-gas-releasing systems [5]. The principal rule is the same- to provide a density lower than the gastric fluids so that they would be capable of floating on the gastric juice in the stomach [6]. Floating of dosage forms can be achieved by the inclusion of a gas generating agent in an inert matrix [7]. Acid-base reactions have been utilized to produce diverse pharmaceutical preparations that effervesce on contact with water. As a result of effervesce and gas generation, density of the system lessens and makes it float on the gastric fluid. Non-gas-releasing systems are generally prepared from one or more matrix forming polymers chosen from polycarbonates, polyacrylates, polymethacrylates, or polystyrene together with a second gel-forming, highly swellable hydrocolloid component, which is typically a cellulose compound or a polysaccharide. Upon contact with gastric fluids, the polymer is hydrated and a colloidal gel network is formed which is directly responsible for the drug release. The air trapped within the swollen polymer allows the buoyancy of the dosage form [7]. Thus, actually, the trapped air grants the polymer a density that is smaller than 1, resulting in buoyancy.

In order to achieve gastric retention, a few requirements should be met. The dosage form must resist premature gastric emptying, i.e., have rapid buoyancy and must be able to withstand forces caused by peristaltic waves. Another requirement is a long duration of floating. In addition, it should dissolve slowly enough to serve as a drug reservoir [8]. Following these requirements it is highly important to rationally select appropriate ingredients to achieve desirable floating behavior and strength. In this context, the selection of a matrix polymer is of a great importance.

Recently, a floating delivery system based on albumin was developed [9]. In contrast to cellulose derivatives, egg albumin has never been used for pharmaceutical applications as a matrix polymer in floating delivery systems. Albumin based solid dosage form can serve as an alternative to the costly soft gelatin capsule formulation, sublingual solutions or the oromucosal spray.

### REFERENCES

[1] Singh B N, Kim K H. 2000. Floating drug delivery systems: an approach to oral controlled drug delivery via gastric retention. J Control Release 63:235-259.
[2] Liu Q, Fassihi R. 2008. Zero-order delivery of a highly soluble, low dose drug alfuzosin hydrochloride via gastro-retentive system. Int J Pharm 348:27-34.
[3] Hwang S J, Park H, Park K. 1998. Gastric retentive drug-delivery systems. Crit Rev The Drug Carrier Syst 15:243-284.
[4] Iannuccelli V, Coppi G, Sansone R, Ferolla G. 1998. Air compartment multiple-unit system for prolonged gastric residence. Part II. In vivo evaluation. Int J Pharm 174:55-62.
[5] Arora S, Ali J, Ahuja A, Khar R K, Baboota S. 2005. Floating drug delivery systems: a review. AAPS PharmSciTech 6:E372-90.
[6] Adibkia K, Hamedeyazdan S, Javadzadeh Y. 2011. Drug release kinetics and physicochemical characteristics of floating drug delivery systems. Expert opinion on drug delivery 8:891-903.
[7] Prinderre P, Sauzet C, Fuxen C. 2011. Advances in gastro retentive drug-delivery systems. Expert opinion on drug delivery 8:1189-203.
[8] Pawar V K, Kansal S, Garg G, Awasthi R, Singodia D, Kulkarni G T. 2011. Gastroretentive dosage forms: a review with special emphasis on floating drug delivery systems. Drug Deliv. 18:97-110.
[9] Rosenzweig O, Lavy E, Gati I, Kohen R, Friedman M. 2013. Development and in vitro characterization of floating sustained-release drug delivery systems of polyphenols. Drug Deliv. 20180-9.

### SUMMARY OF THE INVENTION

There is a growing need for standardized cannabinoid delivery systems as current approved cannabinoid formulations such as buccal spray and oil solutions require frequent administration of immediate release formulations in order to maintain therapeutic levels. At such therapeutic levels side effects associated with the peaks and troughs of immediate release systems may be encountered. Therefore, it is of a great importance to develop oral formulations for prolonged release of cannabinoids. This is especially important for patients suffering from chronic conditions such as multiple sclerosis (MS) plasticity, neuropathic and chronic pain, cancer related pain, epilepsy, autism and other conditions.

In general, poor drug absorption may be associated with an API having low aqueous solubility, poor permeability through the stomach or along the intestine or a narrow absorption window. Cannabinoids have a narrow absorption window at the beginning of the intestinal tract (limited to the small intestine). Since the transient time cannot be extended by pharmaceutical means, to prolong the time of the intestinal absorption phase, there is a need in a delivery system which releases the active compound at the stomach in a controlled release manner. In that way, the active compound slowly moves to the intestine and absorbs there. This type of delivery system diminishes side effects associated with immediate release. Furthermore, the gradual release of the active compound into the upper part of the intestine enhances the bioavailability of the cannabinoids.

Increasing the gastric retention time (GRT) of a drug is a desirable feature for various medical indications, since during floatation of a solid oral dosage form in the gastric environment, the drug can be slowly and gradually released at a desired rate from the solid dosage form, thereby resulting in an increased GRT and allowing for a better control of the fluctuations in plasma drug concentration.

The present invention is based on the finding that cannabinoids are primarily absorbed at the beginning of the intestine and are not likely to be absorbed through the colon (while absorption through other parts of the intestine and colon is a key factor in controlled release formulations). As a result, the classical controlled release tablet that relies on drug absorption through the whole intestine is less suitable. To achieve controlled release in the stomach and absorption primarily in the intestine, the inventors have developed a tablet that remains in the stomach for a period of approximately 8 hours, during which time releases its cargo and allows absorption at the upper portion of the intestine.

The tablet is based on (egg) albumin which contains the active material, e.g., a phyto-cannabinoid, but which does not typically comprise a gas generating agent (GGA). When the albumin matrix absorbs water, it expands and floats, thus, remaining in the stomach for approximately 8 hours. The matrix gradually releases the active material, which becomes absorbed in the upper part of the intestine, exhibiting enhanced bioavailability. Also, entrapment of the tablet in the stomach allows for a prolonged release while utilizing the active, e.g., cannabinoid absorption window.

As floating is achieved by use of albumin, GGA is no longer necessary; thus vacating some of the volume which could be occupied by the GGA, permits loading of the tablet with greater amounts of the active material. As the data provided herein demonstrates, administration of a cannabinoid such as CBD in a tablet form according to the invention demonstrates a five-fold increase in bioavailability as compared to administration of a CBD solution.

Thus, in one of its aspects, the present invention provides a stomach floating solid oral dosage form comprising albumin and at least one cannabinoid, the solid oral dosage form being formulated to permit a continuous release of the at least one cannabinoid and subsequent absorption in the intestine.

The invention further provides a solid oral dosage form comprising albumin (as a matrix forming agent) and at least one cannabinoid. In some embodiments, the dosage form is free of a gas generating agent (GGA) and is further configured for stomach floating. In some embodiments, stomach floating is achievable by compressing the dosage form using a compression force of below 1 ton.

In another of its aspects, the present invention provides a solid oral dosage form comprising albumin and at least one cannabinoid, wherein the oral dosage form optionally contains a gas generating agent (GGA) and wherein the oral dosage form is prepared using a compression force of below 1 ton.

In some embodiments, the oral dosage form is free of a GGA.

The invention further provides a gastro-retentive solid oral dosage form comprising albumin and at least one cannabinoid, wherein the oral dosage form optionally contains a gas generating agent and wherein the oral dosage form is prepared using a compression force of below 1 ton.

In some embodiments, the oral dosage form is free of a GGA.

The solid oral dosage form of the invention is particularly suitable for treatment of diseases and disorders typically treatable by an effective amount of at least one cannabinoid. While the solid oral dosage form is engineered or adapted for prolonged residence in the stomach, i.e., prolonged gastro-retention, the at least one cannabinoid exerts its effect through absorption at a region of the intestine and not via stomach absorption. As used herein, the “solid oral dosageform” is generally a pharmaceutical composition having a solid core (e.g., a tablet core, a capsule core, a pellet core or granulate core) and a coating. Alternatively, the solid oral dosage form may be in the form of a continuous matrix material, e.g., albumin, that encompasses, includes, comprises, embeds or generally holds an effective amount of the active, namely of the at least one cannabinoid material. In accordance with the present invention, the matrix material is not chemically associated with the active material.

In some embodiments, the solid oral dosage form is a tablet. In some embodiments, the solid oral dosage form is a capsule. In some embodiments, the solid oral dosage form is a pellet, and in some other embodiments, the solid oral dosage form is a granule.

The solid oral dosage forms of the invention may be formed into “unit oral dosage forms” which comprise physically discrete units of the solid forms, each unit containing a predetermined quantity of the at least one cannabinoid calculated to produce a desired therapeutic effect, in association with a carrier as described herein, and optionally with any other component of a formulation as described herein. The unit oral dosage form is selected from tablets, caplets, sachets and discrete granules.

In some embodiments, the unit oral dosage form of the present invention comprises between about 50 mg and about 500 mg of the at least one cannabinoid.

As readily recognized by a person of skill in the art, features (e.g., time to float/drug release profile) of the unit solid oral dosage form of the present invention obtained by the herein described methods depend on various parameters such as the type and amount of the matrix forming agent, the formulation variables (composition of amount of additives) and the compression method (e.g., stamping press vs. rotary press, manual vs. automatic punch and die device). Accordingly, a person of skill in the art will know how to fine-tune the desired properties of the unit solid oral dosage form by modifying the above mentioned parameters to obtain a unit solid oral dosage form with the required properties (e.g., floatation time period of between several minutes to several hours or of at least several hours to suit treatment of a specific disease).

The solid oral dosage forms exhibit a short ‘time-to-float’ (or a floating lag time) period. The time-to-float period exhibited by formulations of the invention is between 0.5 to 5 minute(s) as measured from time of contact of the solid oral dosage form with the gastric medium (gastric juices of the stomach) and time of floating. In some embodiments, the time-to-float may be longer. Flotation enables the slow/delayed release of the cannabinoid. Therefore, by increasing the total duration of floating in the gastric environment, an efficient sustained release of the cannabinoid may be obtained.

In some embodiments, the time-to-float is up to 30 seconds, when measured upon contacting a liquid mimicking the gastric environment at an acidic pH; the liquid may be any liquid such as water, saline or simulated gastric fluid, i.e., “U.S. pharmacopeia simulated gastric fluid” which refers to simulated gastric fluid prepared according to U.S. Pharmacopeia 23, with pepsin.

Without wishing to be bound by theory, the albumin used in solid oral dosage forms of the invention acts as a matrix forming agent that forms the solid carrier in which the cannabinoid is encompassed, included, comprised, embedded or generally held. The albumin used for forming the solid oral dosage forms of the present invention may be any albumin known in the art, including serum albumins (e.g., human serum albumin, bovine serum albumin), egg albumin (e.g., ovalbumin) and albumin derived from seeds (e.g., soybean albumin).

In some embodiments, the albumin is egg albumin, which may be obtained from a commercial source or be synthetically prepared (e.g., by expression of recombinant proteins). In some embodiments, the albumin is native egg albumin.

In some embodiments, the albumin consists essentially of ovalbumin (e.g., chicken ovalbumin). In some embodiments, the albumin comprises ovalbumin (e.g., chicken ovalbumin) along with additional egg proteins. In some embodiments, the albumin consist essentially of ovalbumin, i.e., comprises at least 99.1, 99.2, 99.3, 99.5, 99.6, 99.7, 99.8 or 99.9 wt % ovalbumin.

The ovalbumin may be a naturally occurring ovalbumin, i.e., an ovalbumin expressed by an organism and/or in an egg of the organism (e.g. chicken ovalbumin), and/or a protein homologous to a naturally occurring ovalbumin. The ovalbumin may be at least 80% homologous, optionally at least 90% homologous, optionally at least 95% homologous, optionally at least 98% homologous, and optionally at least 99% homologous to a naturally occurring ovalbumin (e.g., chicken ovalbumin).

In some embodiments, the albumin is native albumin, namely albumin which has not been denatured, i.e., albumin which substantially retains its native secondary and tertiary structure. The albumin may optionally be covalently modified, for example, by cross-linking the albumin with a suitable cross-linking agent.

In some embodiments, the matrix forming agent consists or comprises native albumin.

In some embodiments, the albumin is in a form of granules or powder.

In some embodiments, the albumin is in the form of a powder.

In some embodiments, the matrix forming agent consist essentially of albumin, namely comprising nearly completely albumin, i.e., comprises at least 99.1, 99.2, 99.3, 99.5, 99.6, 99.7, 99.8 or 99.9 wt % albumin.

In some embodiments, the albumin, e.g., native albumin, may further comprise at least one additional material. The at least one additional material may be selected amongst polymers, polysaccharides (e.g., sucrose, fructose, glucose, mannitol and sorbitol), flavorings, colorants, thickeners, disintegrants (e.g., crospovidone, crosslinked sodium carboxymethyl cellulose, sodium starch glycolate), fillers, binders (e.g., PVP, crossed linked PVP), glidants (e.g., magnesium stearate, colloidal silicon dioxide, starch, talc), wetting agents, surfactants (e.g., PEG400, PEG3500), antioxidants, metal scavengers, pH-adjusting agents, acidifying agents, alkalising agents, preservatives, buffering agents, chelating agents, stabilizing agents, gas-generating agents (GGA), complexing agents, emulsifying and/or solubilizing agents (e.g., Cremophor® RH 40), absorption enhancing agents, modify release agents, taste-masking agents, humectants, sweetening agents and combinations thereof. The at least one additional material does not negatively affect the solid oral dose features described herein.

In some embodiments, the at least one additional material is a polymer. The polymer may be optionally selected from hydrophilic polymers (e.g., water-soluble polymer) and hydrophobic polymers. Some non-limiting examples of polymers which may be optionally included along with the albumin include polymers such as cellulose derivatives (e.g., ethyl cellulose, methyl cellulose, hydroxypropyl cellulose, hydroxypropylmethylcellulose, hydroxyethyl cellulose, hydroxyethylmethylcellulose, carboxymethyl cellulose); polyacrylamides; (meth)acrylic acid-(meth)acrylate copolymers such as poly(methacrylic acid-co-methyl methacrylate) and poly(methacrylic acid-co-ethyl acrylate) (e.g., Eudragit® L copolymers); poly(ethylene oxide) and copolymers thereof, such as poloxamers (poly(ethylene oxide-co-propylene oxide)); polysaccharides (e.g., alginate, arabinogalactan, chitosan); and proteins (i.e., proteins other than native albumin).

As noted herein, products of the invention do not typically comprise a GGA. Where GGA is present, the GGA may be a mixture of equal amounts of citric acid and sodium bicarbonate. As various and different GGA materials are known, where GGA are excluded according to the invention, such GGA may be any one or more of the specified herein or any of the GGA materials known in the art.

In some embodiments, products of the invention are free of a GGA.

The solid oral dosage form of the present invention is formed by applying compression forces that are uniquely low. In some embodiments, the compression forces used for making the solid oral dosage forms are not higher than 1 ton. In some embodiments, the compression force is between about 0.25 ton and about 1 ton. In other embodiments, the compression force is between 0.25 and 0.7 ton (being about 0.0018-0.005 ton/mm²). In some embodiments, the compression force is between 0.25 and 0.5 ton.

The at least one cannabinoid is any one material of a class of chemical compounds, cannabinoid/cannabinoid agonists/cannabinoid-related compounds, acting with various affinities on the endogenous cannabinoid receptors (CB1 and CB2). The term encompasses the group of ligands that include the endocannabinoids (produced naturally by humans and animals), phytocannabinoids (found in cannabis and some other plants) and synthetic cannabinoids (manufactured artificially). The most notable are tetrahydrocannabinol (THC) and cannabidiol (CBD) as well as synthetic derivatives of phytocannabinoids.

The term also refers to the classical cannabinoids originating from or non-cannabinoids mimicking the natural cannabinoids present in the viscous resin produced in glandular trichomes of a cannabis plant. At least 85 different cannabinoids have been isolated from various strains of cannabis, so far. The main classes of the classical cannabinoids are shown in Table 1 below.

TABLE 1
Main classes of natural cannabinoids

| Type | Skeleton |
|---|---|
| Cannabigerol-type CBG | 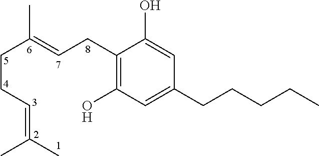 [SMILES: CCCCCc1cc(O)c(C/C=C(\C)CCC=C(C)C)c(O)c1] |
| Cannabichromene-type CBC | 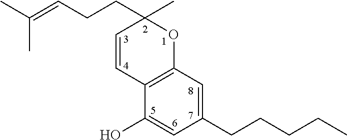 [SMILES: CCCCCc1cc(O)c2c(c1)OC(C)(CCC=C(C)C)C=C2] |
| Cannabidiol-type CBD | 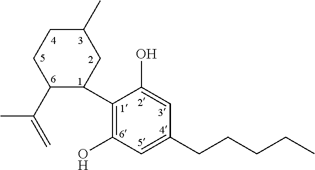 [SMILES: C=C(C)C1CCC(C)CC1c1c(O)cc(CCCCC)cc1O] |
| Tetrahydrocannabinol-and Cannabinol-type THC, CBN | 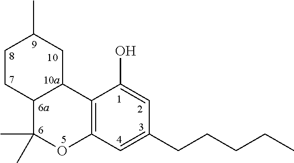 [SMILES: CCCCCc1cc(O)c2c(c1)OC(C)(C)C1CCC(C)CC21] |
| Cannabielsoin-type CBE | 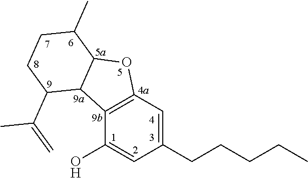 [SMILES: C=C(C)C1CCC(C)C2Oc3cc(CCCCC)cc(O)c3C12] |
| iso-Tetrahydrocannabinol-type iso-THC | 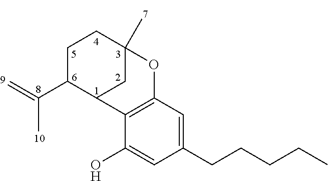 [SMILES: C=C(C)C1CCC2(C)CC1c1c(O)cc(CCCCC)cc1O2] |
| Cannabicyclol-type CBL | 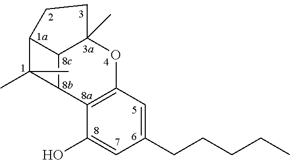 |
| Cannabicitran-type CBT | 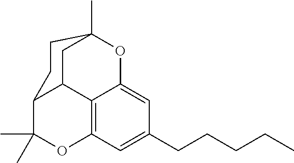 [SMILES: CCCCCc1cc2c3c(c1)OC(C)(C)C1CCC(C)(CC31)O2] |

Thus, in some embodiments of the invention, the solid dosage form may comprise, as an active ingredient, or as a combination of such actives, at least one of a tetrahydrocannabinol (THC), cannabinol-type (CBN), cannabidiol-type (CBD), cannabigerol-type (CBG), cannabichromene-type (CBC), cannabielsoin-type (CBE), iso-tetrahydrocannabinol-type (iso-THC), cannabicyclol-type (CBL), cannabicitran-type (CBT), a derivative, a precursor or a combination thereof. All classes derived from cannabigerol-type compounds and differ mainly in the way this precursor is cyclized. The classical cannabinoids are derived from their respective 2-carboxylic acids (2-COOH, also denoted with —A) by decarboxylation (catalyzed by heat, light, or alkaline conditions).

In some embodiments, the active is tetrahydrocannabinol or cannabidiol acid precursors, THC-A or CBD-A.

In some embodiments, the cannabinoids are selected from tetrahydrocannabinol (THC), cannabidiol (CBD) and cannabinol (CBN). Further selections of cannabinoids that may be used in accordance with the invention are as follows:
THC (Tetrahydrocannabinol, including the two isoforms Δ9-THC, Δ8-THC and the acid form THC-A)
CBD (Cannabidiol and the acid form CBD-A)
CBN (Cannabinol)
CBG (Cannabigerol)
CBC (Cannabichromene)
CBL (Cannabicyclol)
CBV (Cannabivarin)
THCV (Tetrahydrocannabivarin)
CBDV (Cannabidivarin)
CBCV (Cannabichromevarin)
CBGV (Cannabigerovarin); and
CBGM (Cannabigerol Monomethyl Ether).

Thus, in some embodiments, a solid dosage form of the invention may comprise as an active ingredient at least one of THC, THCA, CBD, CBDA, CBN, CBG, CBC, CBL, CBV, THCV, CBDV, CBCV, CBGV, CBGM, a derivative, a precursor, and a combination thereof.

A solid dosage form may comprise each of the components disclosed herein in various amounts. Generally and without being bound by any particular stated amounts, the amount of the albumin, active materials and other components may vary. In a non-limiting example, albumin, e.g., native albumin, is present in an amount of between 30 and 50 wt %, the amount of the at least one cannabinoid may be between 5 and 80 wt %, and the amount of the other additives present may amount to between 0.5 and 30 wt %, as measured relative to the total weight of the solid oral dosage form.

In another one of its aspects, the present invention provides a method of treatment or prevention of a disease or disorder, the method comprising administering to a subject in need thereof a solid oral dosage form of the invention, the form comprising at least one cannabinoid.

The disease or disorder treatable by a solid oral dosage form of the invention is any one clinical conditions that is treatable by cannabinoid/cannabinoid agonists/cannabinoid-related compounds and may include, for example, anorexia, autism, emesis, neuropathic and chronic pain, inflammation, multiple sclerosis, neurodegenerative disorders (such as Parkinson's disease, Huntington's disease, Tourette's syndrome, Alzheimer's disease), epilepsy, spasticity, autism, tuberculosis, inflammatory bowel diseases, including ulcerative colitis and Crohn's disease, irritable bowel syndrome, glaucoma, osteoporosis, schizophrenia, cardiovascular disorders, cancer, obesity, and metabolic syndrome-related disorders, fibromyalgia and graft versus host disease.

In another aspect, the invention provides an oral solid dosage form, according to the invention, prepared by a method comprising compressing a homogeneous mixture of albumin, at least one cannabinoid and optionally a gas-generating agent with a force of between about 0.25 and between about 1 ton to obtain a (monolithic) homogenous unit solid oral dosage.

In some embodiments, the dosage form is prepared by a method comprising obtaining a homogenous mixture of albumin, at least one cannabinoid and optionally a gas-generating agent.

In yet another of its aspects, the present invention provides a method for preparation of a floating solid oral solid dosage form, said method comprising compressing a homogeneous mixture of albumin, at least one cannabinoid and optionally a gas-generating agent with a force of between about 0.25 and between about 1 ton to obtain monolithic homogenous unit solid oral dosage.

In some embodiments, the process comprises obtaining a homogenous mixture of albumin, at least one cannabinoid and optionally a gas-generating agent.

In some embodiments, the method may comprise:
blending albumin with at least one cannabinoid to obtain a homogeneous mixture;
optionally adding a gas-generating agent to the said homogeneous mixture;
compressing said homogeneous mixture with a force of between about 0.25 and between about 1 ton to obtain a homogenous unit solid oral dosage.

In some embodiments, compressing the herein described homogeneous mixture is performed using, e.g., a punch and die device, wherein the pressure applied to the punch is determined according to the area of the unit solid oral dosage form (e.g., tablet) being formed. In some embodiments, the pressure is from about 0.0018 to about 0.005 tons per mm².

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows the results of a THC-CBD-Metoprolol intra-colon administration model in the freely moving rat model.

FIG. 2 shows the results of a study supporting the notion that THC is practically not absorbed through the colon in comparison to PO administration.

FIG. 3 shows the results of a study supporting the notion that CBD is practically not absorbed through the colon in comparison to PO administration.

FIG. 4 shows the results of a CBD dissolution tests in five formulations.

FIG. 5 shows the results of an in-vivo experiment comparing administration of GRCAN tablet-F3 to CBD solution in the freely moving rat model. GRCAN formulation increased CBD oral bioavailability by a five-fold compared to CBD solution.

FIG. 6 shows the dissolution profile of CBD release (% of nominal) from CBD egg albumin tablet in simulated intestinal gastric fluid (mean±SD, n=3).

FIG. 7 shows the dissolution profile of CBD release (% of nominal) from CBD egg albumin/HPMC tablet in simulated intestinal gastric fluid (mean±SD, n=3).

FIGS. 8A-D depict overhead view and side view of CBD-egg albumin tablets (A, B-0 time point, C, D-24h).

FIGS. 9A-D show overhead view and side view of CBD-HPMC tablets (A, B-time point 0, C, D-24h).

FIG. 10 shows dissolution profile of THC release (% of nominal) from THC egg albumin tablet, 1 ton force, in simulated intestinal gastric fluid (mean±SD, n=3).

FIG. 11 shows dissolution profile of THC release (% of nominal) from THC egg albumin tablet, 2 ton force, in simulated intestinal gastric fluid (mean±SD, n=3).

FIG. 12 shows overhead view of THC in Egg albumin 1 ton pressed tablets.

FIGS. 13A-B show overhead view of THC in Egg albumin 2 ton pressed tablets (A-time point 0, B-8 h).

### DETAILED DESCRIPTION OF THE INVENTION

Metoprolol, Polysorbate 20 (Tween® 20) Polysorbate 80 (Tween® 80), Sorbitan monooleate 80 (Span® 80), ethyl lactate, cannabigerol (CBG), Egg albumin (Mw 44 kDa) and ammonium acetate were purchased from Sigma Aldrich (Rehovot, Israel). polyvinylpyrrolidone (PVP), cross-linked polyvinylpyrrolidone and Polyoxyl 40-hydroxy castor oil (Cremophor® RH40) were purchased from BASF The Chemical Company (Ludwigshafen Germany). Lecithin was purchased from Cargill (Minneapolis, MN, USA). Tricaprin (Cremer COOR®; MCT C10-95) was a gift from CREMER Oleo Division (Hamburg, Germany). Acetonitrile (ACN), ethanol, n-hexane, ethyl acetate, hydrochloric acid (HCl) 37% v/v were purchased from J. T. Backer (Phillipsburg, NJ, USA). Sodium bicarbonate and Sodium chloride were purchased from Biolab ltd. (Jerusalem, Israel). Citric acid was obtained from Merck (Darmstadt, Germany). Polyethylene Glycol 400 (PEG 400) and Polyethylene Glycol 3500 (PEG 3500) were purchased from Ofer chemicals lab suppliers (Hod-hasharon, Israel). Hydroxypropylmethyl cellulose (HPMC, Methocel K100M) was obtained from Colorcon (Dartford, England). Pharmaceutical grade THC was purchased from THC Pharm GmbH, The Health Concept, (Frankfurt, Germany). THC was of synthetic origin, with 98% purity. Pharmaceutical grade CBD was purchased from Ai Fame GmbH, (Schonengrund, Switzerland). CBD was plant extracted with 94% purity; related substance THC was less than 0.05%.

### Part I-

### Animals and Surgery

All surgical and experimental procedures were approved by the Animal Experimental Ethics Committee of the Hebrew University, Hadassah Medical School, Jerusalem.

Male Wistar rats (Harlan, Israel) weighing 275-300 g were kept under a 12 h light/dark cycle with free access to food (standard rat chow) and water prior to the procedure. Animals were anesthetized for the period of surgery. An indwelling cannula was placed in the right jugular vein of each animal for systemic blood sampling and tunneled beneath the skin. To simulate colonic delivery, we inserted a cannula directly to a rat caecum as a means to bypass the stomach and small intestine. Both cannulas were exteriorized at the dorsal part of rats' neck. After completion of the surgical procedure, the animals were transferred to individual cages to recover overnight (12-18 h). During this recovery period, they had free access to food and water. On the day of experiment, they were deprived of food for 4 hr prior to drug administration, but not water. Throughout the experiments, free access to food was available 4 h post oral administration.

### Experimental Protocol

Lipid-Based Formulation The lipid-based formulation used for this study was a self-nano emulsifying formulation previously developed by this group. Briefly, ethyl lactate and lecithin were placed in a vial at a ratio of 4:1, respectively. The mixture was heated to 40° C. until completely dissolved. Then, tricaprin, Cremophor® RH40, Tween® 20, and Span® 80 were added at the ratio of 1:1:1:1. The mixture was stirred and heated to 40° C. until a homogeneous solution was formed. THC, CBD, and metoprolol were dissolved in this solution at 2.67% w/w, 2% w/w and 2.67% w/w respectively. Upon dispersion in pre-heated water (1:9 v/v), this composition self-emulsified into o/w nano dispersion. The resulting nano particles dissolved in their lipid core the lipophilic THC and CBD. While metoprolol was most probably dissolved in the aqueous phase of the dispersion. Metoprolol was used as a positive control for colonic absorption.

### Colonic and Oral Administration of THC, CBD and Metoprolol

On the day of experiment, animals were divided into two groups. The experimental group (n=6) received dispersed THC-CBD-metoprolol formulation through the colonic cannula and water via an oral feeding tube. The control group (n=5), received the THC-CBD-metoprolol formulation orally and water through the colonic cannula corresponding to volume of drug-formulation administered in the experimental group. Animals in both groups received THC 20 mg/kg, CBD 15 mg/kg and metoprolol 20 mg/kg. Systemic blood samples (0.30 ml) were obtained from the intravenous cannula. To prevent dehydration, equal volumes of physiological saline were administered to the rats following each blood sampling. Sequential blood samples were collected into heparin-containing test tubes at predetermined time intervals. Plasma was separated by centrifugation (3220 g, 10 min, 4° C.) and stored at −20° C. pending analysis.

### Experimental Protocol of CBD Relative Bioavailability Study

### Gastro Retentive CBD Tablets

Tablets were prepared by direct compression using a 5 mm die, manually pressed with a 1-ton force for 30 s. Each tablet was designed to weigh 70 mg. Tablet measurements used, have been previously demonstrated by our group, to be big enough in order to remain in a rat stomach and provide gastric retention. In-vitro dissolution was performed in USP simulated gastric fluid under fasted conditions (pH 1.2) with 5% v/v Tween® 80. The buffers were prepared without enzymes. For the dissolution test, each glass tube contained 100 ml, heated to 37° C.±3. Rotation speed was 100 rpm. Following tablet placing in the dissolution glasses, time was measured until tablet began to float. A sample of 100 μl was taken in each time point and replaced with a fresh buffer. Results were adjusted according to this minor dilution. Time points for the experiment in simulated gastric fluid were 1, 2 3, 4, 5,6,7,8 and 24 h. Samples were analyzed via an HPLC-UV method. HPLC-UV conditions were as follows: Luna C-8(2), 5 μm, 150×4.6 mm, 100 Å column (Phenomenex, CA, USA). An isocratic mobile phase of 5 mM NaH2PO4 in water (pH 3.0) and acetonitrile at 30:70 ratio, at flow rate of 1 ml/min, 40° C.±5. UV Monitoring wavelength: 220 nm. CBD RT was 7.7 min. Linearity for CBD was between 0.5-500 μg/ml with R²>0.999.

Relative Oral Bioavailability of CBD in Solution Vs. GR-CAN Tablet

Experimental group (n=3) received CBD in GR-CAN tablet. The control group (n=3), received CBD in a propylene glycol: ethanol: water solution (4.5:4.5:1) at a 3 mg/ml concentration.

Animals were randomly assigned to the experimental groups. THC, CBD, and metoprolol were dissolved in lipid based vehicle at 2.67% w/w, 2% w/w and 2.67% w/w respectively. THC and CBD were the model molecules tested for colonic absorption and metoprolol served as the control. Metoprolol is a molecule known for its colonic absorption; it has several marketed controlled release formulations and is important in this experiment as proof for the validity of the surgery. All animals underwent the same surgery. On the day of experiment, they were divided into two groups. The experimental group (n=6) received THC-CBD-MET formulation diluted in water (1:10 v/v) through the colonic cannula and water PO. The control group (n=5), received the THC-CBD-MET formulation PO and water through the colonic cannula corresponding to volume of drug-formulation administered in the experimental group. Animals in both groups received THC 10 mg/kg, CBD 15 mg/kg and metoprolol 15 mg/kg. Systemic blood samples (0.35 ml) were obtained by the intravenous cannula, placed in the jugular vein. Samples were taken at 5 min pre-dose and at different time points post dose; according to the pharmacokinetic profile: 0, 20 min, 40 min, 1 hr, 1.5 hr, 2 hr, 4 hr, 6 hr, 8 hr, 12 hr and 24 hr. To prevent dehydration, equal volumes of physiological solution are administered to the rats following each blood sampling. Plasma was separated by centrifugation (4000 rpm, 7 minutes, 4° C.) and stored at −20° C. pending analysis.

### Plasma Assay

Plasma CBD, THC and Metoprolol concentrations were determined using HPLC-MS. Plasma aliquots of 150 μL were spiked with 10 μL of internal standard cannabigerol (CBG; 1 μg/mL). ACN (200 μL) was added to each test tube (tubes A) and vortex-mixed for 1 min. The extraction of CBD, THC, Metoprolol and CBG was performed by ethyl acetate (3 mL) that was added to each test tube (tubes A), followed by 1 min. vortex-mixing. After centrifugation at 4000 rpm for 10 min, the ethyl acetate organic layer was transferred to fresh glass test tubes (tubes B) and evaporated to dryness (Vacuum Evaporation System, Labconco, Kansas City, MO). Then, tubes B were reconstituted in 80 μL of ACN: water (80:20). The resulting solution (80 μl) was injected into the HPLC-MS system.

### PK Analysis

The concentration vs. time data and pharmacokinetic parameters such Tₘₐₓ, Cₘₐₓ, and AUC were calculated using non-compartmental analysis with WinNonlin® (version 5.2, Pharsight, Mountain View, CA).

### Statistical Analysis

All values are expressed as mean±standard error of the mean (SEM) if not stated otherwise. To determine statistically significant differences among the experimental groups, student t-test was used. P value of less than 0.05 was termed significant.

### Gastro Retentive Cannabidiol Tablets

Tablets were prepared by direct compression using a 5 mm die, manually pressed with a 1 ton force for 30 sec. Each tablet is designed to weigh 70 mg (Table 2). Tablet measurements used have been previously demonstrated by our lab, to be big enough in order to remain in a rat stomach and provide gastric retention.

In-vitro dissolution was preformed USP simulated gastric fluid under fasted conditions (pH=1.2) with 5% Tween 80. The buffers were prepared without enzymes. For the dissolution test, each glass tube contained 100 mL, heated to 37° C.±3. Rotation speed was 100 rpm. Following tablet placing in the glasses, time was measured until tablet began to float. 100 μL sample was taken in each time point and replaced with a fresh buffer. Results were adjusted according to this minor dilution. Time points for the experiment in simulated gastric fluid were 1, 2 3, 4, 5, 6, 7, 8 and 24 hr. Samples were analyzed via an HPLC-UV method.

TABLE 2
Composition (% w/w) and floating properties of GRCAN tablets

| | | | | | | | | | | Cross | Floating |
|---|---|---|---|---|---|---|---|---|---|---|---|
| | | Egg | Citric | Sodium | Mg | PEG | PEG | Cremophor | | linked | lag time |
| Form. | CBD | albumin | acid | bicarbonate | stearate | 3500 | 400 | RH 40 | PVP | PVP | (FLT, min) |
| F1 | 14.3 | 44.7 | 15 | 15 | 1 | | | | 10 | | <5 |
| F2 | 14.3 | 44.7 | 15 | 15 | 1 | | 5 | 5 | | | <5 |
| F3 | 14.3 | 44.7 | 15 | 15 | 1 | | 3 | 7 | | | <0.8 |
| F4 | 14.3 | 44.7 | 15 | 15 | 1 | | | 10 | | | <2 |
| F5 | 14.3 | 44.7 | 15 | 15 | 1 | | 10 | | | | <5 |
| F6 | 14.3 | 44.7 | 15 | 15 | 1 | | | | | 10 | <0.5 |
| F7 | 14.3 | 44.7 | 15 | 15 | 1 | 10 | | | | | <5 |
| F8 | 14.3 | 44.7 | 15 | 15 | 1 | 2 | | 8 | | | <0.66 |
| F9 | 14.3 | 44.7 | 15 | 15 | 1 | | | | 5 | 5 | <1 |

### Relative Oral Bioavailability of CBD in Solution Vs GRCAN Tablet

Male Wistar rats (Harlan, Israel) weighing 275-300 g were kept under a 12 h light/dark cycle with free access to food (standard rat chow) and water prior to the procedure. Animals were anesthetized for the period of surgery. An indwelling cannula was placed in the right jugular vein of each animal for systemic blood sampling and tunneled beneath the skin as described before.

The experimental group (n=3) received CBD in GRCAN tablet. The control group (n=3), received the CBD in a propylene glycol:ethanol:water solution (4.5:4.5:1) at a 3 mg/ml concentration. Animals in both groups received, CBD 30 mg/kg and. Systemic blood samples (0.35 ml) were obtained by the intravenous cannula, placed in the jugular vein. Samples were taken at 5 min pre-dose and at different time points post dose; according to the pharmacokinetic profile: 0, 0.5 hr, 1 hr, 2 hr, 3 hr, 4 hr, 5 hr, 6 hr, 7 hr, 8 hr and 12 hr. To prevent dehydration, equal volumes of physiological solution are administered to the rats following each blood sampling. Plasma was separated by centrifugation (4000 rpm, 7 minutes, 4° C.) and stored at −20° C. pending analysis.

### Plasma Assay

Plasma aliquots of 150 μL were spiked with 10 μL of internal standard cannabigerol (CBG; 1 μg/mL). ACN (200 μL) was added to each test tube (tubes A) and vortex-mixed for 1 min. The extraction of CBD was performed by N-hexane (3 mL) that was added to each test tube (tubes A), followed by 1 min. vortex-mixing. After centrifugation at 4000 rpm for 10 min, the N-hexane organic layer was transferred to fresh glass test tubes (tubes B) and evaporated to dryness (Vacuum Evaporation System, Labconco, Kansas City, MO). Then, tubes B were reconstituted in 80 μL of ACN: water (80:20). The resulting solution (80 μl) was injected into the HPLC-MS system.

### Results

### THC-CBD-Metoprolol Intra-Colon Administration in the Freely Moving Rat Model

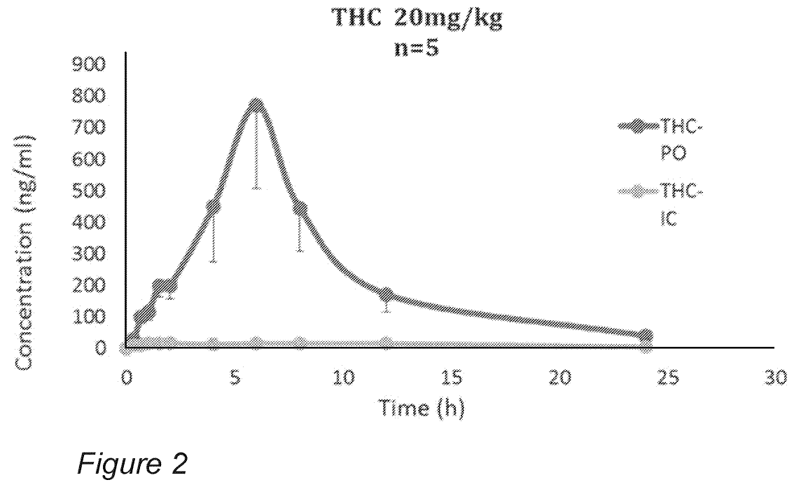

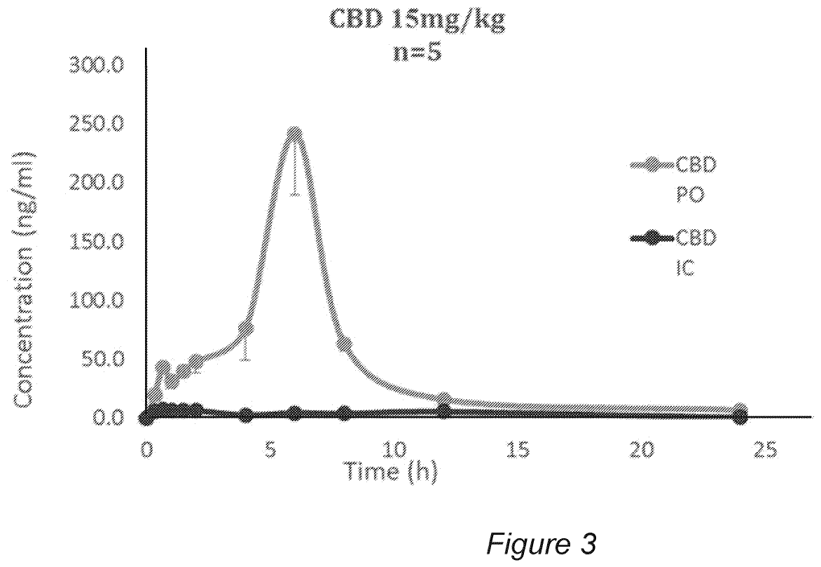

When comparing the PK profile obtained from both groups, we can see that THC and CBD are practically not absorbed through the colon in comparison to PO administration (FIGS. 2 and 3). Metoprolol administration through the colon resulted in approximately 4-fold higher AUC in comparison to metoprolol PO administration.

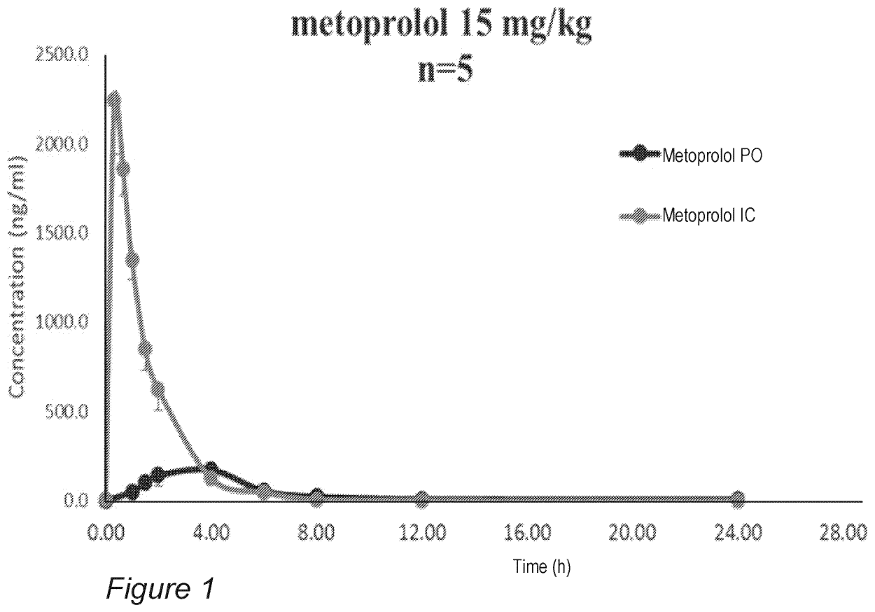

These results prove that THC and CBD have minimal colonic absorption, thus the controlled release device of the invention has the limited absorption time of 6-8 hr or the alternative of a gastric retentive dosage form (FIG. 1).

### Gastro Retentive Cannabinoid (GRCAN)

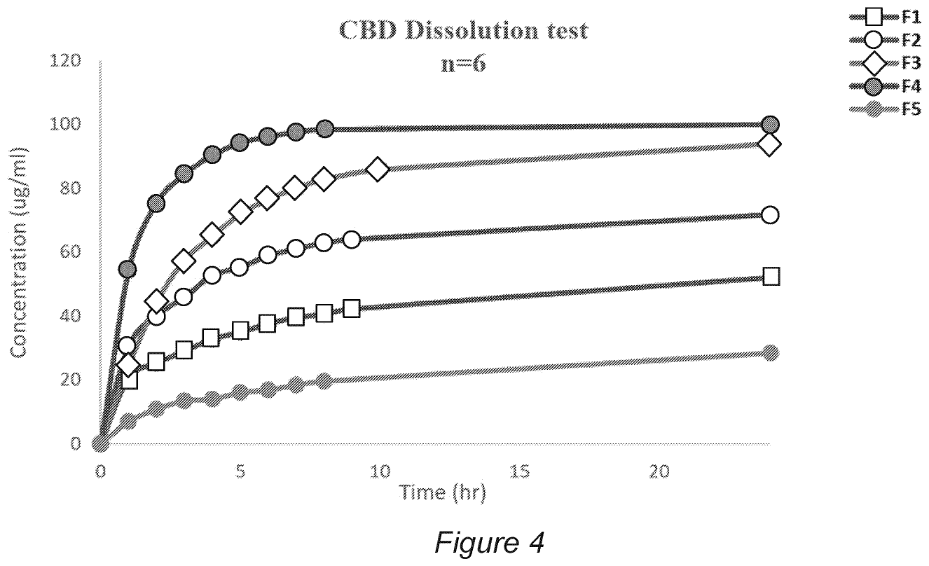

As shown in FIG. 4, all tested formulations resulted in a floating time of less than 5 min. Formulation 3 was selected for in-vivo experiments since it was composed of a less Cremophor RH40 and thus was less friable.

### In-Vivo Investigation of CBD-GRCAN Tablet

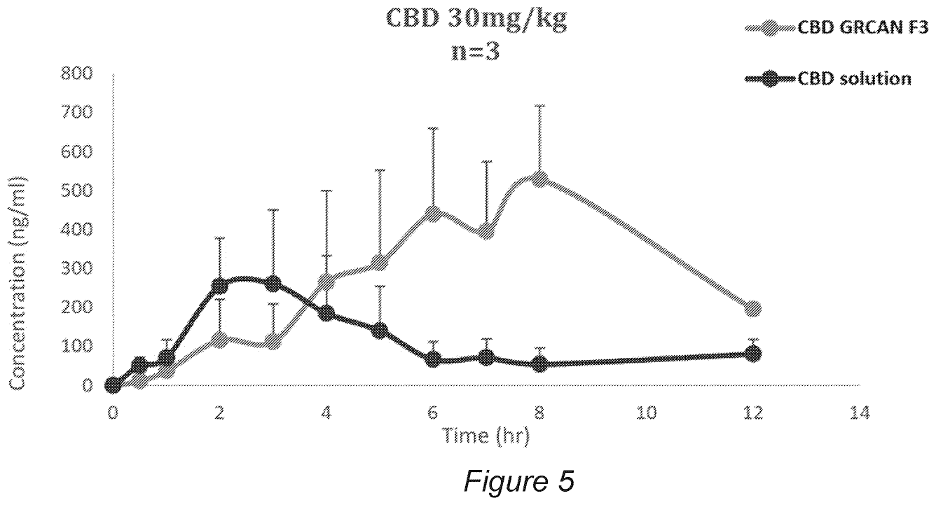

In-vivo experiment compared administration of GRCAN tablet-F3 to CBD solution in the freely moving rat model. GRCAN formulation increased CBD oral bioavailability by a five-fold compared to CBD solution (FIG. 5, Table 3).

TABLE 3
AUC values (mean ± SEM) obtained following PO administration
of CBD in solution and GRCAN tablet. CBD dose 30 mg/kg
(n = 3 for each group) (*) A significant difference
(p < 0.05) from CBD corresponding values was found.

| | [symbol] (h*ng/ml) |
|---|---|
| solution | [symbol] 70 |
| GRCAN tablet | [symbol] 828(*) |

[symbol]

indicates data missing or illegible when filed

### Part II-

### Tablet Preparation:

### CBD Tablets

The respective powders were blended thoroughly with a mortar and pestle.

Tablets were prepared by direct compression using an 18×10 mm oval punch die, manually pressed with a 1-ton force for 30 sec. Each tablet is designed to weigh 500 mg (Table 4).

THC tablets

We dissolved the THC and Cremophor in 300 μl of ethanol and placed at 40′C until fully dissolved. The solution was dripped over the egg albumin using a Pasteur pipette. The vial was washed with another 250 μl of ethanol and added as well, After granulation the powder was placed at 40° C. for 40 minutes to evaporate the ethanol. Then an accurate amount of silica was added until the powder gained sufficient flowing properties. Tablets were prepared by direct compression using a 20×7 mm oval-flat punch die, manually pressed with a 1-ton or 2-ton force for 30 sec. Each tablet was designed to weigh approximately 580 mg (Table 5).

TABLE 4
Composition (% w/w) and floating properties of CBD-GRCAN tablets

| | | | | | | Floating | |
|---|---|---|---|---|---|---|---|
| | | Egg | | Magnesium | Cremophor | lag time | Floating |
| Tablet | CBD | albumin | HPMC | stearate | RH 40 | (FLT, min) | Time (h) |
| T1 | 20 | 69 | — | 1 | 10 | <0.5 | 10 |
| T2 | 20 | 69 | — | 1 | 10 | <0.5 | 10 |
| T3 | 20 | 69 | — | 1 | 10 | <7 | 8 |
| T4 | 20 | 50 | 20 | 1 | 9 | — | — |
| T5 | 20 | 50 | 20 | 1 | 9 | — | — |
| T6 | 20 | 50 | 20 | 1 | 9 | — | — |

TABLE 5
Composition (% w/w) and floating properties of THC-GRCAN tablets

| | | | | | | | Floating | |
|---|---|---|---|---|---|---|---|---|
| | | Egg | | Magnesium | Cremophor | Press | lag time | Floating |
| Tablet | THC | albumin | Silica | stearate | RH 40 | (Ton) | (FLT, min) | Time (h) |
| T1 | 2.6 | 94.5 | 0.5 | 1 | 1.4 | 1 | <5 | 1 |
| T2 | 2.6 | 94.5 | 0.5 | 1 | 1.4 | 1 | <0.5 | 1 |
| T3 | 2.6 | 94.5 | 0.5 | 1 | 1.4 | 1 | <0.5 | 1 |
| T4 | 2.6 | 94.5 | 0.5 | 1 | 1.4 | 2 | — | — |
| T5 | 2.6 | 94.5 | 0.5 | 1 | 1.4 | 2 | — | — |
| T6 | 2.6 | 94.5 | 0.5 | 1 | 1.4 | 2 | — | — |

### Dissolution Test:

In-vitro dissolution was performed in a USP simulated gastric fluid under fasted conditions (pH=1.2) with 5% Tween 80. The buffers were prepared without enzymes. For the dissolution test, each glass tube contained 250 mL, heated to 37° C.±3. Rotation speed was 150 rpm. Following tablet placing in the glasses, time was measured until tablet began to float (FLT). 200 μL sample was taken in each time point and replaced with a fresh buffer. Results were adjusted according to this minor dilution. Time points for the experiment in simulated gastric fluid were 0.25, 0.5, 1, 2 3, 4, 5,6,7,8 (for THC), 10 and 24 hr (for CBD). Samples were analyzed via an HPLC-UV method.

### Sample Analysis in HPLC-UV

Analytical test for CBD and THC content was conducted using HPLC-UV.
Column used: Luna C-8(2), 5 m, 150×4.6 mm, 100 Phenomenex, 00F-4249-E0
Mobile phase: 5 mM NaH2PO4 in water pH 3.0: Acetonitrile at 30:70 ratio.
Diluent: Acetonitrile: water=30:70
Column temperature: 40° C.±5° C.
Sample Temperature: 10° C.±5° C.
UV Monitoring wavelength: 211 nm
CBD RT: 7.7 min
THC RT: 12.7 min

### Results

### Dissolution Test-CBD Release

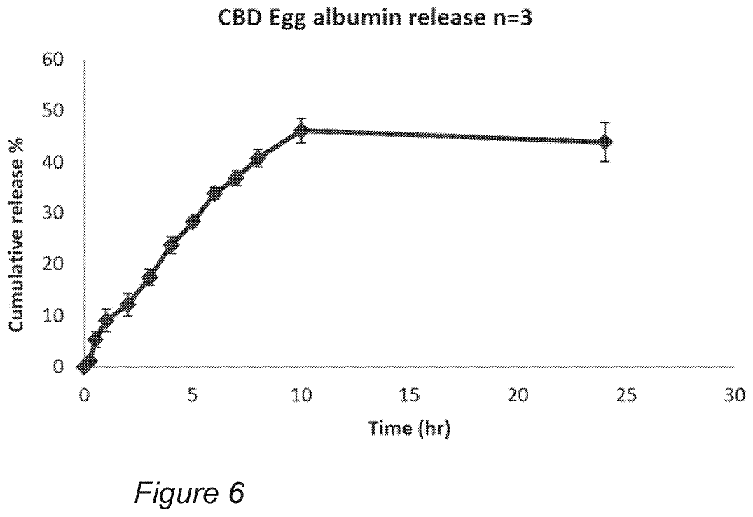

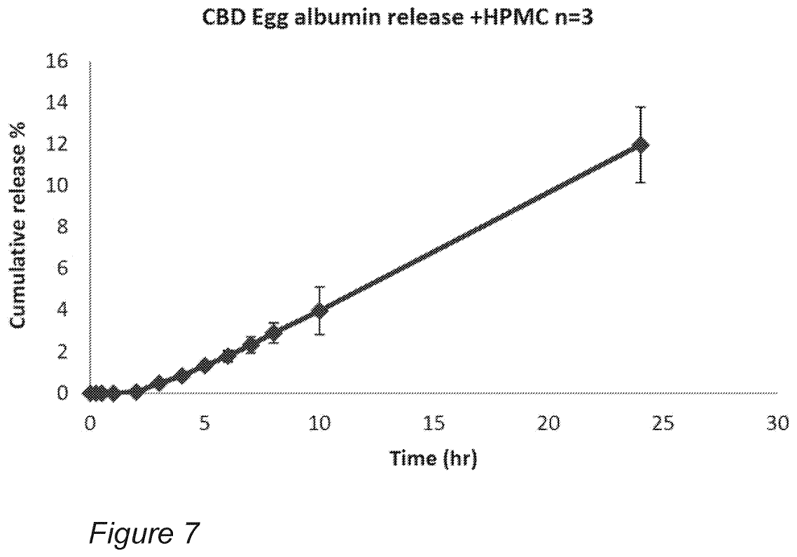

Each tablet tested contained theoretically 100 mg of CBD. Thus, in a 250 mL dissolution glass, the max concentration achieved should have been 400 ug/mL. This concentration is set as the theoretical concentration. Results are presented as % of CBD released from nominal concentration. FIG. 6 depicts release of CBD from egg albumin based tablet. After 10 h, approximately 46% is released. Tablets 1-3, floated for at least 8 hr. FIG. 7 depicts release of CBD from an egg albumin and PMC tablets. These tablets (4-6) did not float and resulted in a 1200 release over 24 hr.

### Changes in Size

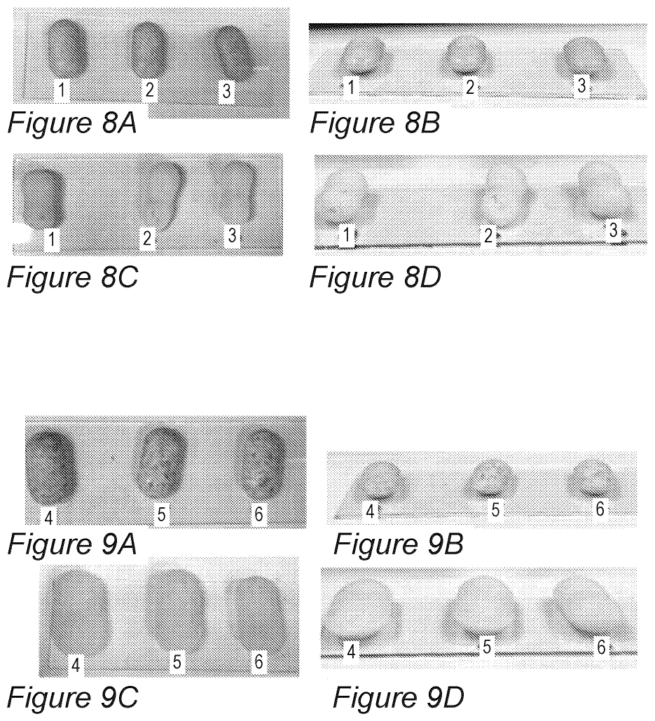

Tablet measurements are composed of length, width and height. With these parameters we calculated volume of tablets before the dissolution test and after the last sample of 24 hr. Egg albumin tablets (tablets 1-3), resulted in a particular shape with the middle of tablet narrower than the sides. As a result, we calculated the max/min width and max/min height. In average, tablets increased 1.7 fold after 24 hr (FIG. 8).

Egg albumin and HPMC tablets increased in use uniformly (tablets 4-6). In average, tablets increased 5.2 fold after 24 hr (FIG. 9).

### Dissolution Test-THC Release

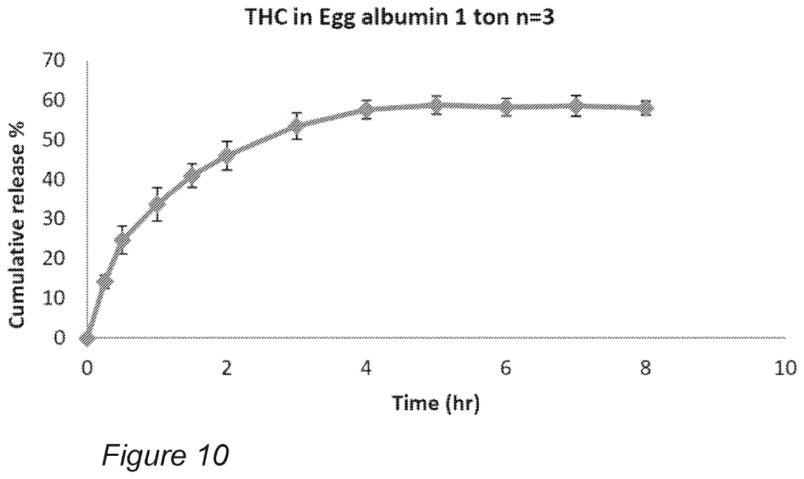

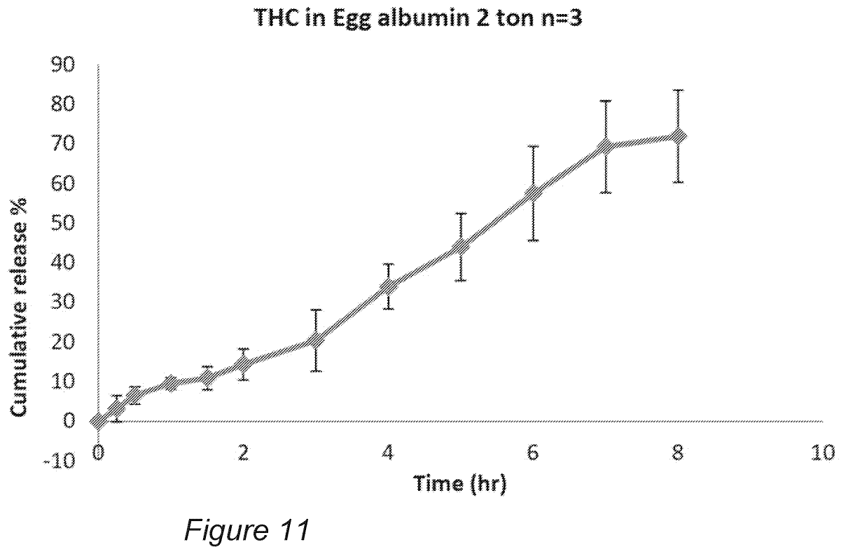

Each tablet tested contained theoretically 15 mg of THC. Thus, in a 250 mL dissolution glass, the max concentration achieved should have been 60 μg/mL. This concentration is set as the theoretical concentration. Results are presented as % of THC released from nominal concentration. FIG. 10 depicts release of THC from egg albumin based tablet pressed at 1 ton. After 10 h, approximately 60% is released. Tablets 1-3, floated for 1 hr and then disintegrated. FIG. 11 depicts release of THC from an egg albumin pressed at 2 ton. These tablets (4-6) did not float and resulted in a 72% release over 8 hr. 4 hr from the beginning of the experiment, tablets were split down the middle and two parts remained intact till the end of the trial-8 hr.

### Changes in Size

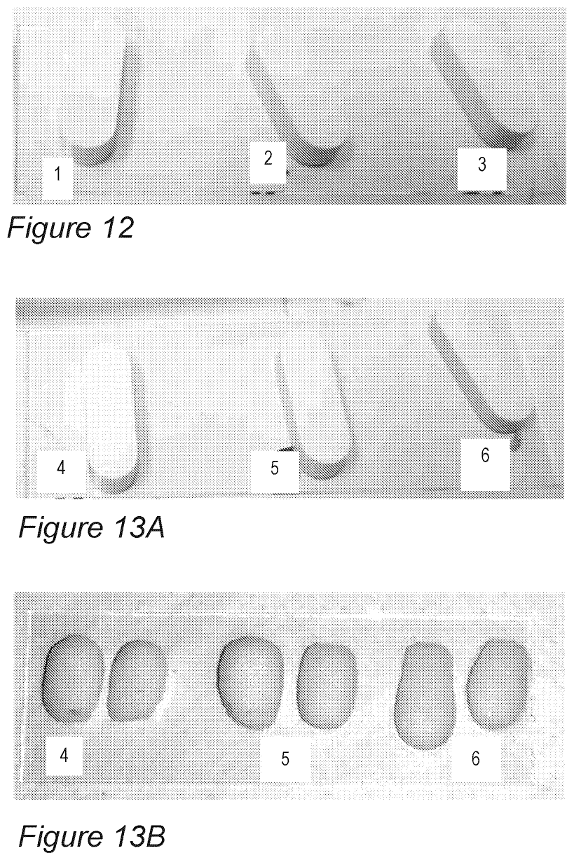

Tablet measurements are composed of length, width and height. With these parameters we calculated volume of tablets before the dissolution test. Egg albumin tablets, pressed at 1 ton (tablets 1-3, FIG. 12), disintegrated after 1 hr. As a result, size changes after 8 hr were not documented. Egg albumin tablets, pressed at 2 ton (tablets 4-6, FIGS. 13A and 13B) were divided by the middle after 4 hr. The two resulting halves were measured for the three mentioned parameters. In average, if both parts are taken into consideration, tablets increased 1.5 fold after 8 hr.

TABLE 7
Changes in THC tablet size parameters: length, width
and volume. Change in volume is expressed in ratio

| | | Length (mm) | | | Width (mm) | | | Height (mm) | | Volume (mm³) | | |
|---|---|---|---|---|---|---|---|---|---|---|---|---|
| Time | 0 | | 8 h | 0 | | 8 h | 0 | | 8 h | 0 | 8 h | Ratio |
| T1 | 20.4 | | | 7.2 | | | 4.2 | | | 616.2 | | |
| T2 | 20.6 | | | 7.2 | | | 4.4 | | | 650.5 | | |
| T3 | 20.4 | | | 7.2 | | | 4.3 | | | 631.4 | | |
| T4 | 20.3 | 17.8 | | 7.2 | 8.9 | | 4.0 | 4.7 | | 577.3 | 736.7 | 1.3 |
| | | 9.3 | 8.5 | | 4.7 | 4.2 | | 2.5 | 2.2 | | | |
| T5 | 20.4 | 19.00 | | 7.1 | 10.8 | | 3.9 | 4.7 | | 572.7 | 959.7 | 1.7 |
| | | 9.9 | 9.1 | | 5.7 | 5.1 | | 2.5 | 2.2 | | | |
| T6 | 20.2 | 22.4 | | 7.1 | 9.8 | | 3.1 | 3.0 | | 447.8 | 667.3 | 1.5 |
| | | 12.6 | 9.8 | | 4.9 | 4.9 | | 1.6 | 1.4 | | | |
| Mean | | | | | | | | | | | | 1.5 |

Consumption of medicinal cannabis is divided into several routs of administration. The most prevalent route is inhalation or smoking of whole plant. Smoking results in rapid onset of absorption and effect. However, this route has health disadvantages, inter-subject variability alongside a biased opinion from society and regulation. Oral medicinal cannabis products are often based on oils such as Marinol®, a sesame oil synthetic THC capsule or recently approved Epidiolex®, a sesame oil-ethanol oral solution of CBD. Although these products have a relatively slower onset of effect, they require more than a single administration per day. A prominent marketed product is the orumucosal spray Sativex®, a THC-CBD formulation of propylene glycol and ethanol. Patients use the spray several times a day for the relief of neuropathic pain and spasticity symptoms of MS. Frequent use of the spray often causes mouth ulcerations and lesions. These examples from clinical practice show that there is a need for developing cannabinoid CR formulations that will treat to patients needs and ultimately increase compliance and adherence.

Currently, the most leading technology for gastro retentive dosage form of cannabinoids is based on an accordion pill that unfolds in stomach, thus avoiding gastric emptying, enabling drug release for a longer period compared to the control Sativex.

The rational for developing a gastro retentive tablet is based on preserving the use of upper intestine for increased and prolonged absorption, while developing a solid dosage form with a less costly, more easily upscaled technique.

In classical oral controlled release (CR) formulations, the absorption phase of a drug is prolonged beyond the small intestine. The transit time from the small intestine to the caecum (the first part of the large intestine) is approximately 4 h. The time interval in the small intestine is too short for controlled release dosage forms, unless the drug can be equally absorbed from the large intestine. Thus, the release profile for most oral CR dosage forms can be effective for about 6-8 h if taking into consideration transit time from the mouth to the caecum. For a drug which can be absorbed from the large intestine, the time interval for absorption can be increased to 1 day. Therefore, a prerequisite condition for a successful CR dosage form is sufficient absorption from the colon. To investigate regional absorption of THC and CBD from the colon, compounds were administered directly to rat cecum via a specially inserted cannula. Regional colonic absorption was compared to systemic absorption, following an oral (PO) bolus, which encompasses absorption from the upper parts of the intestine. For this experiment a concept termed “absorption cocktail approach” was used. In this method, target molecules are administered together with standard probes that aid in understanding drug absorption processes, absorption kinetics, PK etc. The standard molecule used for colonic absorption was metoprolol. Metoprolol is a compound with sufficient absorption through the entire intestinal tract.

Plasma exposure of metoprolol following colonic administration was higher compared to PO administration. There are reports suggesting a considerable intestinal first-pass extraction of metoprolol in rats, evaluated via intraduodenal administration. It may be possible that bypassing the small intestine may have enabled the compound to avoid intestinal first pass metabolism it undergoes, thus resulting in increased absorption. Contrary to metoprolol, both THC and CBD resulted in poor absorption through the colon, compared to their oral administration. The use of metoprolol demonstrated that low colonic absorption of cannabinoids is not result of the procedure of colon cannulation, but outcome of their physicochemical properties. These results are in line with researchers' hypothesis that lipophilic molecules as cannabinoids have poor absorption from the colon, which is not physiologically designed for the absorption of fats or lipids.

In light of these preliminary results regarding cannabinoid narrow absorption window, the CR formulation of gastro-retentive dosage form was chosen. Different technologies are implemented in order maintain the dosage form in the stomach and overcome the physiological tendency to evacuate stomach content. At the center of this manuscript is the development of a floating gastro retentive dosage form based on egg albumin, carbon dioxide generating compounds and CBD as the active compound. A screening of excipients was conducted in order increase of drug release over at least 8 h. Lowest results are seen with HPMC, PEG3500 and PEG400, about 25-30% drug release while tablets that contained 10% surfactant Cremophor® RH40, resulted in approximately 98% drug release. Alongside drug release, the use of a surfactant in the formulation is of importance on account of CBD's poor solubility. However, this formulation was also too friable for in-vivo investigation. Thus, a series of formulations was evaluated, based on this surfactant with decreased concentration. Formulation based on PEG400 and Cremophor® RH40 (3:7) was investigated in-vivo in the freely moving rat model. This composition was selected since it had sufficient drug release after 8 h (˜80%) and a combination of excipients that may have aided in CBD solubilization in the stomach. The suitability of the rat model for gastro retentive dosage forms, was previously demonstrated by Stepensky et al. (2001) which proved that tablets of the used dimensions remain in rat stomach for at least 8 h. This was proven by a series of X-ray images of specially marked tablets. Although, the ultimate evaluation of a gastro retentive dosage form ought to be conducted in a clinical trial, the rat model sheds light on potential drug candidates and formulation before moving towards a clinical setting.

In vivo results in the freely moving rat model demonstrate the potential of the gastro retentive dosage form in prolonging drug absorption phase. As anticipated, Tₘₐₓ was delayed for the GR-CAN tablet to 8 h, compared to 2 h for the solution. Not only was the drug plasma concentration profile prolonged, but exposure and relative bioavailability were also increased compared to solution. This may be result of the CBD's physicochemical properties which deem the compound with dissolution rate limited absorption. CBD is a Biopharmaceutical Classification System (BCS) class 2 molecule that although has sufficient permeability through the intestinal wall, its dissolution in the aqueous environment of the stomach is a rate limiting step. As a result, upon gradual release of smaller amounts from the tablet matrix, with the aid of tablet surfactants, the dissolution is under “sink conditions” and the absorption is less dependent on this process. In addition, both Cremophor® RH40 and PEG400 were reported as excipients with inhibitory effect on phase I and phase II enzymes. Indeed, CBD's poor oral bioavailability is result of low solubility in the GI tract and susceptibility to extensive pre systemic metabolism. Thus, the addition of Cremophor® RH40 and PEG400 may have aided in decreasing intestinal metabolism and increasing bioavailability.

Thus, this invention concerns a controlled release dosage form for the highly lipophilic cannabinoids. The lipophilic nature of cannabinoids renders the molecules unsuitable candidates for conventional CR formulations that require colonic absorption. As estimated by researchers, lipophilic cannabinoid colonic absorption is low due physiological role of the colon to absorb mainly water and minerals and avoid lipid absorption. Regional absorption of cannabinoids has not been investigated, particularly in comparison to systemic absorption, emphasizing the importance of this work. The narrow absorption window of cannabinoids places these compounds as candidates for GRDF that utilize absorption from the upper small intestine while retained in the stomach.

1-32. (canceled)

33. A solid oral dosage form comprising albumin and at least one cannabinoid, wherein the oral dosage form is free of a gas generating agent (GGA) and wherein the oral dosage form is prepared using a compression force of below 1 ton.

34. The oral dosage form according to claim 33, being in a form selected from a tablet, a capsule, a pellet and a granulate.

35. The oral dosage form according to claim 33, having a stomach floatation time period of at least several hours.

36. The oral dosage form according to claim 33, having a time-to-float period of between 0.5 to 5 minutes, or below 0.5 minutes, as measured from time of contact of the solid oral dosage form with the gastric medium to time of floatation.

37. The oral dosage form according to claim 33, wherein the albumin is selected from serum albumins, egg albumin and albumin derived from seeds.

38. The oral dosage form according to claim 37, wherein the seed albumin is soybean albumin.

39. The oral dosage form according to claim 33, wherein the albumin is in a form of granules or powder.

40. The oral dosage form according to claim 33, wherein the albumin being in a form of a matrix material in which the at least one cannabinoid is carried.

41. The oral dosage form according to claim 40, wherein the matrix material further comprising at least one additional material.

42. The oral dosage form according to claim 41, wherein the at least one additional material is selected amongst polymers, polysaccharides, flavorings, colorants, thickeners, disintegrants, fillers, binders, glidants, wetting agents, surfactants, antioxidants, metal scavengers, pH-adjusting agents, acidifying agents, alkalising agents, preservatives, buffering agents, chelating agents, stabilizing agents, gas-generating agents (GGA), complexing agents, emulsifying and/or solubilizing agents, absorption enhancing agents, modify release agents, taste-masking agents, humectants, sweetening agents and combinations thereof.

43. The oral dosage form according to claim 42, wherein the at least one additional material is a polymer.

44. The oral dosage form according to claim 43, wherein the polymer is selected from hydrophilic polymers and hydrophobic polymers.

45. The oral dosage form according to claim 33, being formed by applying compression forces between about 0.25 ton and about 1 ton, or between 0.25 and 0.7 ton, or between 0.25 and 0.5 ton.

46. The oral dosage form according to claim 33, wherein the at least one cannabinoid a cannabinoid/cannabinoid agonists/cannabinoid-related compound acting on an endogenous cannabinoid receptors (CB1 or CB2).

47. The oral dosage form according to claim 46, wherein the at least one cannabinoid is selected from THC (Tetrahydrocannabinol), CBD (Cannabidiol), CBN (Cannabinol), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin) and CBGM (Cannabigerol Monomethyl Ether).

48. A method of treatment or prevention of a disease or disorder, the method comprising administering to a subject in need thereof a solid oral dosage form according to claim 33.

49. A method for preparation of a floating solid oral solid dosage form, said method comprising compressing a homogeneous mixture of albumin, at least one cannabinoid and optionally a gas-generating agent with a force of between about 0.25 and about 1 ton to obtain a homogenous unit solid oral dosage.

50. The method according to claim 49, the method comprising:
blending albumin with at least one cannabinoid to obtain a homogeneous mixture;
optionally adding a gas-generating agent to the said homogeneous mixture;
compressing said homogeneous mixture with a force of between about 0.25 and between about 1 ton to obtain monolithic homogenous unit solid oral dosage.

51. The method according to claim 49, wherein the pressure is from about 0.0018 to about 0.005 tons per mm².

52. A sustained or prolonged release oral dosage form comprising albumin and at least one cannabinoid, wherein the oral dosage form optionally containing a gas generating agent and wherein the oral dosage form is prepared using a compression force of below 1 ton.
